(19) United States
(12) Patent Application Publication
(10) Pub. No.: US 2005/0256459 A1
(43) Pub. Date: Nov. 17, 2005

(54) Device and method for securing a catheter to a patient

(75) Inventors: Pamela Howard, Allentown, PA (US); Eben Howard, Allentown, PA (US); Steven C. Walker, Waxahachie, TX (US); John M. Shepherd, San Antonio, TX (US)

Correspondence Address: CAHN & SAMUELS LLP, 2000 P STREET NW, SUITE 200, WASHINGTON, DC, 20036, US

(73) Assignee: Eben Howard and Pamela A. Howard, Allentown, PA (US)

(21) Appl. No.: 11/108,132

(22) Filed: Apr. 18, 2005

Related U.S. Application Data
(63) Continuation of application No. 60/563,007, filed on Apr. 19, 2004.

(51) Int. Cl. A61M 5/32

(52) U.S. Cl. 604/174

(57) ABSTRACT

The invention is a hub anchor for securing a catheter to a patient's tissue or skin. The hub anchor preferably includes in at least one embodiment includes a body having a pair of walls connected by an activation mechanism having at least one leg in sliding communication with each of the walls. In an engaged state, the legs penetrate a patient's skin and cause the hub to clamp the catheter to the patient. The invention also includes a method of placing the hub over a catheter to be secured and pressing the activation mechanism to pierce the patient's skin with the legs to secure the hub and the catheter to the patient.

This application claims the benefit of U.S. provisional application Ser. No. 60/563,007, filed on Apr. 19, 2005, which is incorporated herein by reference.

### I. FIELD OF THE INVENTION

The present invention generally relates to catheters and more specifically to devices for anchoring catheters to a patient.

II. BACKGROUND OF THE INVENTION

A variety of devices can be employed in human and veterinary medicine to deliver medication to a particular site in an attempt to benefit a patient. For example, a medical catheter can be used to deliver medication to a patient's brain or spinal cord in an attempt to treat a malady, disease, or illness. Devices can also be employed to administer fluids or nutrients to a patient. For example, a medical IV can be used to deliver fluids through the skin of a dehydrated patient. In addition to the above uses, various devices can also be inserted into a patient to withdraw blood, to measure pressures within the blood vessel or to allow the passage of various instruments through the vessel (for example, a pacemaker wire). Regardless of the employed device, however, it must be securely attached to a patient, either internally (for example, into organ tissue) or to the skin.

Regardless of the reason for inserting the device, maintaining controlled placement of the device in the position of interest is highly desirable. In some instances, the delivered drugs are highly concentrated but are more effective if they are precisely delivered to the position of interest.

Further, in some instances, only a small portion of a highly concentrated drug is required to be therapeutically beneficial to the patient. Thus, failure to maintain controlled placement of the device in the position of interest (that is, dislodgement of the device) may be detrimental to the patient by causing her/him to receive more than the required amount of the highly concentrated drug.

In addition to the previously described side effects of dislodgement, accidental migration to an undesired location can cause bleeding, infection, collapse of a lung, heart rhythm abnormalities, and other potentially fatal complications. Regardless of whether such complications ensue, such accidental migration or dislodgment can require repositioning or replacing the catheter, which can waste invaluable time and can subject the patient to further risks and possibly additional trauma and punctures.

Several methods have been used in attempts to secure catheters more reliably. One method commonly employed to immobilize a catheter is that of simply using sections of adhesive tape to secure the catheter such as a medical IV to the skin of a patient. The taping method can have obvious drawbacks. For example, it is cumbersome to use and remove the tape. Further, the tape is often rigid and does not move with the compliance modulus of the skin. Thus, it is difficult to use this method with a patient who is restless.

Another method employs a pair of plastic wings with each wing having an eyelet. The catheter is inserted into the patient, up to the hub. The medical professional then passes a needle and thread through one of the eyelets and then through the patient's skin. Finally, the medical professional ties a knot, cuts the thread, and repeats the process on the opposite eyelet. The sewing technique is not entirely reliable, as the wings often break, especially if tension is applied to cinch the catheter. The pressure exerted on the skin is highly variable and suturer dependent. Too little pressure may result in a loose, floppy attachment which allows the catheter to slide in and out of its insertion site, with the dangerous consequences described above. Too much pressure, however, may cause skin necrosis and breakdown, which may cause a persistent ulcer, infection, and/or the undesirable effects of dislodgement.

Moreover, suturing can be tedious and time-consuming. Many small parts such as the needle and thread are often handled. This can increase the chances that one of these parts will be dropped off the sterile field and contaminated.

Straight needles are generally employed for the suturing process because they eliminate the cost of a curved needle and the instrument to hold it (a needle driver). As a result, however, the skin must be awkwardly pinched, and the suturing process itself can be more traumatic.

Finally, there is also a risk of penetrating too deeply with the needle, and puncturing a vital structure, or even the catheter itself. These problems occur when attempting to attach a catheter-grasping device to a patient who is not motionless. When the patient is unable or unwilling to remain motionless long enough for the catheter-grasping device to be attached, this procedure can become even more difficult and prone to error.

Notwithstanding the usefulness of the existing catheter securing devices, a need exists for a more dependable and safer catheter securing device that can be employed to easily secure a catheter to a patient and prevent dislodgement of the catheter.

### III. Summary of the Invention

The present invention addresses the need for a more dependable and safer catheter securing device. The securing or anchoring device of the present invention can be employed to easily secure a catheter, for example, to a patient to prevent dislodgement of the catheter.

In at least one embodiment, the present invention includes an anchor for a catheter having a hub, the hub anchor including means for securing the catheter hub against the patient, and means for anchoring the hub anchor into the patient.

In at least one embodiment, the present invention includes a device for anchoring a catheter to a patient, the device having an activation mechanism, a pair of walls connected by the activation mechanism, each of the walls having at least one anchoring leg depending therefrom, and the activation mechanism and the walls forming a passageway for the catheter.

In at least one embodiment, the present invention includes a device for securing a catheter hub to a patient, the device including an activation mechanism having a plurality of anchoring legs depending therefrom, a body in communication with the anchoring legs and having a channel extending therethrough between the anchoring legs.

In at least one embodiment, the present invention relates to a securing or anchoring device for a catheter. The securing or anchoring device can be securely fitted over a catheter hub in an intimate manner. The device includes legs that penetrate a patient's tissue or skin to cause the device to secure or anchor the catheter to the patient's tissue or skin, thereby preventing dislodgement of the catheter.

In at least one embodiment, the present invention includes a body having a passageway extending throughout the body for allowing a device (for example, a catheter hub) to pass through and at least two walls. An activation mechanism is coupled to the body. The activation mechanism includes at least two legs. When pressure is applied to the activation mechanism, it interlocks with the body such that the legs penetrate the tissue or skin of a patient, thereby preventing dislodgement of the catheter. To assist in preventing dislodgement of the legs into the tissue or skin of the patient, hooks or spikes can be located on the legs. In certain embodiments, the hooks or spikes include dissolvable material that would allow them to dissolve into the patient's tissue or skin. In particular embodiments of the present invention, each wall includes a first side and a second side wherein the first side is separated from the second side by a cavity. In such embodiments, the legs of the activation mechanism protrude from the cavities and penetrate the tissue or skin of the patient to secure the catheter to the patient.

In other embodiments of the present invention, the securing or anchoring device includes a body having a passageway extending throughout the body and an activation mechanism atop the body. In these embodiments, however, the activation mechanism and the body may not be separate components. Operation of these embodiments of the present invention is similar to operation of the embodiments briefly described above.

An object of at least one embodiment of the present invention is to prevent dislodgement of a catheter from tissue or skin.

Another object of at least one embodiment of the present invention is to increase the dependability and safety of securing catheters to tissue or skin.

Another object of at least one embodiment of the present invention is to minimize the difficulty in the process of securing catheters to tissue or skin.

An advantage of at least one embodiment of the present invention is decreased time involved in securing the catheter to tissue or skin.

Given the following enabling description of the drawings, the device and method of the present invention should become evident to a person of ordinary skill in the art.

### IV. BRIEF DESCRIPTION OF THE DRAWINGS

The use of cross-hatching within these drawings should not be interpreted as a limitation on the potential materials used for construction of the invention. Like reference numerals in the figures represent and refer to the same element or function.

FIGS. 1A-1D illustrate a variety of views of an exemplary embodiment of the invention.

FIGS. 2A and 2B depict different exemplary embodiments of an anchoring leg according to the invention.

FIG. 3 illustrates an end view of an exemplary embodiment of the invention.

FIG. 4 depicts a side view of an exemplary embodiment of an anchoring mechanism according to the invention.

FIGS. 5A and 5B illustrate exemplary embodiments of a body according to the invention.

FIGS. 6A and 6B depict top views of exemplary embodiments of a body according to the invention.

FIG. 6C illustrates a cross-section taken at 6C-6C of FIG. 6B according to the invention.

FIG. 7 depicts an end view of an exemplary embodiment according to the invention.

FIGS. 8A and 8B illustrate end and side views, respectively, of an exemplary embodiment according to the invention.

FIGS. 9A and 9B depict an exemplary embodiment according to the invention.

FIG. 10 illustrates an exemplary embodiment according to the invention.

### V. DETAILED DESCRIPTION OF THE INVENTION

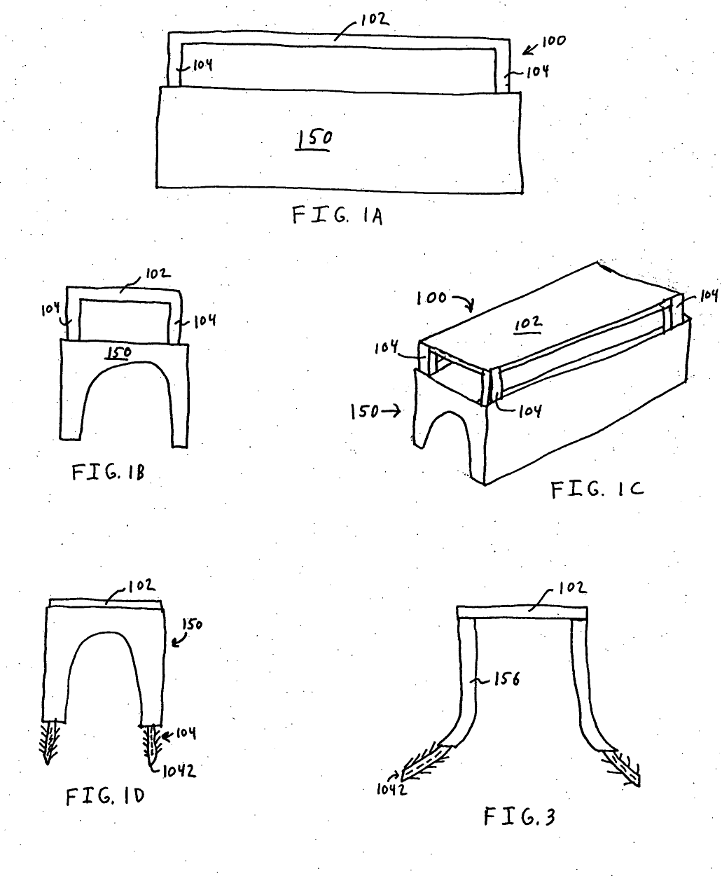

The invention is a catheter hub anchor having an anchoring mechanism and a body (or frame) that serve as an anchor for securing catheters such as an IV to a patient's skin as illustrated, for example, in FIG. 1A-1D. The anchoring mechanism preferably includes a plurality of anchoring legs that pass through the body, which fits around the catheter.

FIGS. 1A-1D illustrate an exemplary embodiment of the invention. FIGS. 1A-1C illustrate the device in an open state, while FIG. 1D illustrates the device in an interlock state (or engaged state).

In FIGS. 1A-1C, the illustrated anchoring mechanism 100 includes an activation mechanism (or button or push surface) 102 with at least two anchoring legs 104 depending from it. Although the activation mechanism 102 is illustrated as rectangular, it-may take a variety of shapes including circular, oval, and square or any other viable shape. The illustrated exemplary embodiment shows an anchoring leg 104 in each corner of the activation mechanism 102. However, there can be a plurality of anchoring legs 104 along each longitudinal side of the activation mechanism 102 or two anchoring legs 104 with one each in two opposing corners of the anchoring mechanism 102.

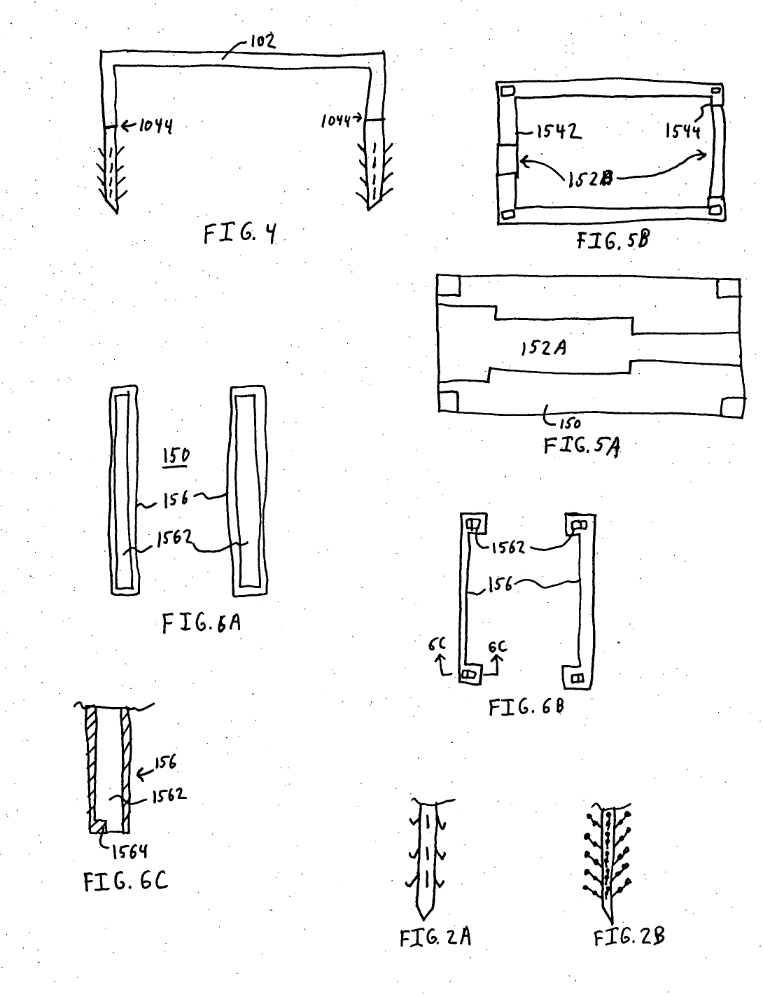

Each anchoring leg 104 includes an end (or tip) 1042 capable of piercing a patient's tissue or skin upon application of a force on the activation mechanism 102. Examples of piercing ends 1042 include an angled end like that shown similar to the end of a needle (FIG. 1D), a conical end (FIGS. 9A and 9B), and a beveled end similar to a cutting instrument such as a scalpel. Along a portion beginning from the piercing end 1042 are a plurality of hooks (FIG. 2A), spikes (FIG. 1D), barbs (FIG. 2B), or any other viable securing mechanisms 1046 for allowing the anchoring legs 104 to remain lodged in the tissue or skin of the patient. The anchoring legs 104 as depicted in FIG. 1D are straight but may also be made of a flexible material such that during insertion they curve out and away from the body 150 as shown in FIG. 3, which illustrates an exemplary embodiment of the body 150 with two side walls 156. The anchoring legs 104 as illustrated also include a portion without securing mechanisms that will act as a stop 1044 to insertion in addition to the activation mechanism being stopped by the body as illustrated, for example, in FIG. 4.

The anchoring legs 104 may be made from a variety of materials including multiple types of material. For example, the anchoring leg 104 may be made of metal with the securing mechanism 1046 made from a biodegradable material such that after a period of time the activation mechanism 102 may be lifted and the anchoring legs 104 removed from the patient's tissue or skin as the securing mechanism 1046 will have dissolved. Or alternatively, the entire anchoring leg 104 may be made, for example, from biodegradable material or metal.

The body 150 as illustrated, for example, in FIGS. 1B-1D and 5A-6B includes a channel (or passageway) through which the catheter hub passes. The body 150 can be any means for stabilizing the catheter hub. The body 150 as illustrated in FIG. 5A includes a channel 152A with a plurality of sections with curved cross-sections to fit a variety of catheter hubs as catheter hubs typically have a larger cross-section at the connection point for an external hose as opposed to closer to the insertion end of the catheter, but one of ordinary skill in the art will appreciate that the channel 152 may be formed to fit different shaped catheter hubs. The channel 152A may have perpendicular channels (not shown) extending from the central channel 152A to be compatible with eyelets present on current catheters.

As illustrated in FIG. 5B, the body 150 includes end walls 1542, 1544 and an open cavity 152B as the channel 152. At least one wall 1542 includes an opening to fit around a portion of the catheter in place of an eyelet wing. Alternatively, the body 150 may include a fluid, gel filled, or foam member providing a channel 152 that is able to conform to the shape of the catheter hub. Examples of acceptable fluid include saline, air including inert gas or any other fluid that is commonly used to fill medical device balloons.

An exemplary embodiment is illustrated in FIGS. 6A and 6B, which shows the body 150 having two side walls 156 each with at least one passageway 1562, which as illustrated in FIG. 6A has a cross-section running substantially the length of the side wall 156, through which the anchoring legs 104 pass. The side walls 156 in at least one embodiment form a channel 152 in which the catheter hub can sit. As illustrated in FIG. 6B, each passageway 1562 may be sized to fit one anchoring leg 104. One of ordinary skill in the art will appreciate based on this disclosure that a wide variety of geometric shapes may be used as the cross-section for the anchoring legs and the passageways. FIG. 3 illustrates another exemplary embodiment where the bottom of the wall passageway is curved out to assist with the insertion of the anchoring legs. As illustrated in FIG. 6C, the passageway may include a narrower opening at the bottom to act as a stop 1564 against insertion of the anchoring leg. These illustrated walls 156 can be combined with the exemplary embodiments shown in FIGS. 5A and 5B and discussed above.

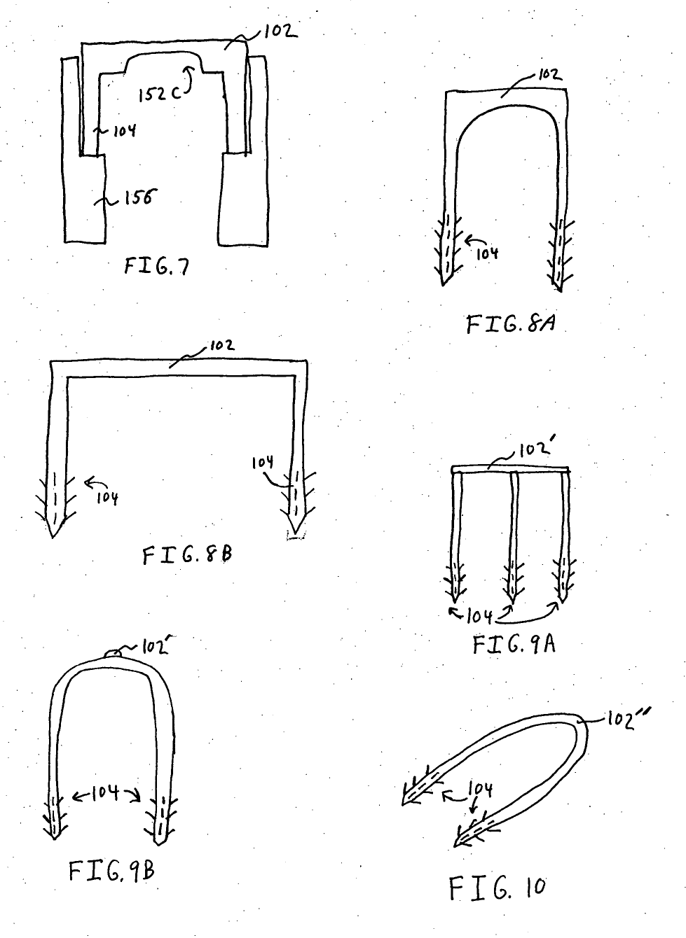

FIG. 7 illustrates an exemplary embodiment where the anchoring mechanism 100 includes a channel 152C formed on the bottom of the activation mechanism 102. The activation mechanism 102 is in sliding engagement with the body 150 such that the walls 156 of the body 150 are not connected directly by another body part but instead are connected through the activation mechanism 102. The walls 156 included shorten passageways along their sides for the anchoring legs 104 to pass through so that the activation mechanism 102 may be lowered sufficiently to have the channel 152C contact the catheter hub and thus secure the catheter against the patient's tissue or skin.

FIGS. 8A and 8B illustrate another exemplary embodiment where the activation mechanism 102 and the body are integrally formed with each other such that as the device is coupled to the catheter hub, the anchoring legs 104 are driven into the patient's tissue or skin as part of the coupling process. FIG. 8B also illustrates an exemplary embodiment where the activation mechanism 102 holds the catheter hub in place after the anchoring legs 104 are inserted into the patient.

FIGS. 9A and 9B illustrate another exemplary embodiment where the securing device includes a plurality of connected pairs of anchoring legs 104 that are driven into the patient's tissue or skin by application of force upon the activation mechanism (or connecting member) 102′ that connects the plurality of anchoring leg 104 pairs. Alternatively, the connecting member 102′ may be omitted and each anchoring leg pair is inserted individually by hand or mechanical means with the material connecting the two anchoring legs 104 considered the activation mechanism 102″.

Another way to describe the invention is in terms of functional language. The hub anchor includes a means for securing the catheter hub against the patient and means for anchoring the hub anchor into the patient. The structure corresponding to the securing means includes the various exemplary embodiments discussed above having a channel 152 (means for providing a channel to fit around the catheter hub), the side walls 156, the end walls 1542, 1544, and the activation means 102 when integrally formed with the body. The anchoring means further can include means for attaching to the patient and means for driving the attaching means into the patient. The structure corresponding to the attaching means includes the various exemplary embodiments for the anchoring legs and in some exemplary embodiments the passageways 1562 and walls 156. The structure corresponding to the driving means includes the activation mechanism 102, 102′, 102″ and their various exemplary embodiments.

After a catheter is inserted, the hub anchor 100 is placed over the catheter hub such that the device and the catheter hub become coupled or at least make contact. The user then presses down on the activation mechanism 102 to drive (or press) the anchoring legs 104 into the patient's tissue or skin. As pressure is applied against the activation mechanism 102, the anchoring legs 104 pierce the patient's tissue or skin and become anchored therein. The body 150 acts as a stop against the activation mechanism 102 from inserting the anchoring legs too far into the patient. The securing mechanism on the anchoring legs then resist removal (or dislodgement) of the device and the catheter from the patient. The whole process is quick compared to the old ways of suturing or taping the catheter hub into place on the patient, along with requiring a lower level of skill than the skill needed for suturing.

The exemplary and alternative embodiments described above may be combined in a variety of ways with each other. Furthermore, the steps and number of the various steps illustrated in the Figures may be adjusted from that shown.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

Although the present invention has been described in terms of particular embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

1. A device for securing a catheter hub to a patient, the device comprising:
an activation mechanism having a plurality of anchoring legs depending therefrom,
a body in communication with said anchoring legs and having a channel extending therethrough between said anchoring legs.

2. The device according to claim 1, wherein each of said anchoring legs includes a piercing tip.

3. The device according to claim 2, wherein each of said anchoring legs includes a plurality of anchors.

4. The device according to claim 2, wherein each of said anchoring legs includes a plurality of hooks.

5. The device according to claim 2, wherein each of said anchoring legs includes a plurality of barbs.

6. The device according to claim 2, wherein each of said anchoring legs includes a plurality of spikes.

7. The device according to claim 1, wherein said body includes structure forming the channel to fit around the catheter hub.

8. The device according to claim 1, wherein said body includes a pair of walls, each of the walls include at least one channel through which at least one of said anchoring legs is in sliding engagement.

9. The device according to claim 8, wherein said channels are curved away from the passageway.

10. The device according to claim 1, wherein said anchoring legs include biodegradable material.

11. A device for anchoring a catheter to a patient, the device comprising:
an activation mechanism,
a pair of walls connected by said activation mechanism, each of said walls having at least one anchoring leg depending therefrom, and
said activation mechanism and said walls forming a passageway for the catheter.

12. The device according to claim 11, further comprising a means for providing a channel fitted to the catheter hub, said channel means located in the passageway.

13. The device according to claim 12, further comprising a second pair of walls connected to both of said first pair of walls, and each of said second pair of walls having an opening passing therethrough.

14. The device according to claim 11, wherein each wall includes at least two anchoring legs depending thereform.

15. The device according to claim 14, wherein each anchoring leg includes a plurality of spikes extending from said anchoring leg towards said activation mechanism.

16. The device according to claim 14, wherein each anchoring leg includes a plurality of securing mechanisms.

17. An anchor for a catheter having a hub, said hub anchor comprising:
means for securing the catheter hub against the patient, and
means for anchoring said hub anchor into the patient.

18. The hub anchor according to claim 17, wherein said anchoring means includes
means for attaching to the patient, and
means for driving the attaching means into the patient.

19. The hub anchor according to claim 18, wherein the securing means includes means for providing a channel to fit around the catheter hub.

20. A method for using the hub anchor of claim 18 comprising:
inserting the catheter into a patient,
placing the hub anchor of claim 18 over the catheter hub,
pushing on the driving means to attach the hub anchor of claim 18 to the patient.
